(19) United States
(12) Patent Application Publication
(10) Pub. No.: US 2013/0196000 A1
(43) Pub. Date: Aug. 1, 2013

(54) COMBINATION THERAPY INCLUDING ISOPHOSPHORAMIDE MUSTARD, ANALOGS, OR SALTS THEREOF

(71) Applicant: Ziopharm Oncology, Inc., Boston, MA (US)

(72) Inventors: Lawrence EINHORN, Indianapolis, IN (US); Jonathan LEWIS, Fairfield, CT (US); Hagop YOUSSOUFIAN, West Roxbury, MA (US)

(73) Assignee: ZIOPHARM ONCOLOGY, INC., Boston, MA (US)

(21) Appl. No.: 13/753,975

(22) Filed: Jan. 30, 2013

Related U.S. Application Data
(60) Provisional application No. 61/593,060, filed on Jan. 31, 2012.

(51) Int. Cl. A61K 31/7048; A61K 31/555; A61K 31/664

(52) U.S. Cl. CPC A61K 31/7048; A61K 31/664; A61K 31/555
USPC 424/649; 514/27

(57) ABSTRACT

In one aspect, a method for treating a subject having a hyperproliferative disorder is disclosed, including administering to the subject a composition including: IPM, an IPM analog, or a pharmaceutically acceptable salt thereof in the dosage from about 70 mg/m²/day to about 160 mg/m²/day; etoposide in the dosage up to about 100 mg/m²/day; and one or more of carboplatin, cisplatin, oxaliplatin, picoplatin, or a combination thereof in the dosage of from AUC 2 mg/mL/min to AUC 7 mg/mL/min; wherein the treatment does not result in a dose limiting toxicity.

### RELATED APPLICATIONS

This application claims priority to U.S. Provisional Application No. 61/593,060, filed on Jan. 31, 2012, and is related to international application numbers PCT/US2005/038523, PCT/US2008/004449, PCT/US2009/052295, all of which are incorporated by reference in their entirety.

### FIELD

This invention relates to methods of treating hyperproliferative disorders. More specifically, this invention relates to combination therapies including isophosphoramide mustard (IPM), analogs, or salts thereof and other anti-cancer agents. More specifically, the composition disclosed herein includes, without limitation, IPM-tris salt.

### BACKGROUND

Lung cancer is a leading cause of cancer-related mortality worldwide and accounts for as many deaths as colorectal, breast, and prostate cancers combined. See, Jemal et al., “Global cancer statistics”, CA Cancer J. Clin. 2011, 61, 69-90. Approximately 13% of patients who receive a diagnosis of lung cancer will have small cell histology. See, Govindan, et al., “Changing epidemiology of small-cell lung cancer in the United States over the last 30 years: Analysis of the Surveillance, Epidemiologic, and End Results database,” J. Clin. Oncol. 2006, 24, 4539-4544. Small cell lung cancer (SCLC) is an aggressive tumor that is characterized by rapid growth and early dissemination, such that the majority of patients present with extensive-stage disease that has spread beyond the ipsilateral hemiothorax. As a result, patients with SCLC usually have a short life expectancy. SCLC is very sensitive to first-line chemotherapy, with response rates that range from 60% to 80%. However, most patients with extensive-stage disease quickly develop chemotherapy-resistant tumors and have a median overall survival of just 8 to 13 months from diagnosis. See, Demedts et al., “Treatment of extensive-stage small cell lung carcinoma: current status and future prospects,” Eur. Respir. J. 2010, 35, 202-215.

Chemotherapy for extensive-stage SCLC includes the combination of a platinum agent and etoposide. Mascaux et al., “A systematic review of the role of etoposide and cisplatin in the chemotherapy of small cell lung cancer with methodology assessment and meta-analysis,” Lung Cancer, 2000, 30, 23-36. Etoposide in combination with cisplatin (EP) has been used in patients with SCLC. See, Sundstrøm et al., “Cisplatin and etoposide regimen is superior to cyclophosphamide, epirubicin, and vincristine regimen in small-cell lung cancer: results from a randomized phase III trial with 5 years' follow-up,” J. Clin. Oncol. 2002, 20(24), 4665-4672; Roth et al., “Randomized study of cyclophosphamide, doxorubicin and vincristine versus etoposide and cisplatin versus alternation of these two regimens in extensive small-cell lung cancer: a phase III trial of the Southeastern Cancer Study Group,” J. Clin. Oncol. 1992, 10, 282-291.

Nevertheless, despite decades of clinical investigation, the 5-year survival for all patients with SCLC remains dismally low, i.e., about 5%, even with the platinum agent-etoposide combination therapy and other chemotherapies currently used. Additionally, standard platinum agent-etoposide combination therapy results in the patient having at least moderate toxic adverse effect. See, Okamoto, et al.: “Randomised phase III trial of carboplatin plus etoposide vs split doses of cisplatin plus etoposide in elderly or poor-risk patients with extensive disease small-cell lung cancer: JCOG 9702,” Br. J. Cancer, 2007, 97, (ii), 162-9. Therefore, there remains a need to develop new and improved chemotherapy for patients with SCLC.

### SUMMARY

Disclosed herein are methods and compositions for combination therapy for treating a subject having a hyperproliferative disorder. The method includes administering to the subject a composition including: isophosphoramide mustard (IPM), an analog thereof, or a pharmaceutically acceptable salt thereof; etoposide and/or teniposide; and one or more of carboplatin, cisplatin, picoplatin, and oxaliplatin. In some embodiments, a subject having a hyperproliferative disorder with an Eastern Cooperative Oncology Group (ECOG) performance status of 2 or better at the start of treatment may be treated with the method and compositions described herein. The ECOG performance status may be described in more detail in the Experimental section.

In some embodiments, the method includes treating the subject with a composition including IPM, an analog thereof, or a pharmaceutically acceptable salt thereof in the dosage from about 70 mg/m²/day to about 160 mg/m²/day; etoposide and/or teniposide in the dosage up to about 100 mg/m²/day; and one or more of carboplatin, cisplatin, picoplatin, and oxaliplatin in the dosage of from AUC 2 mg/mL/min to AUC 7 mg/mL/min; wherein the treatment does not result in a dose limiting toxicity.

In some embodiments, IPM (isophosphoramide mustard, Formula Ia, Scheme 1) or an IPM salt (Formula I, Scheme 1) may be used in the composition for treating a subject with hyperproliferative disorder.

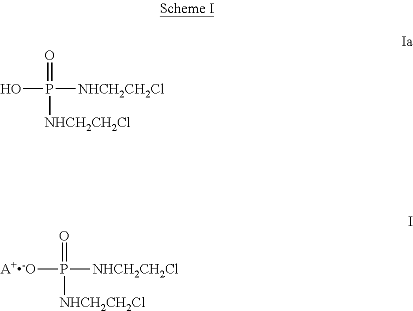

In Scheme I, A⁺ represents an ammonium salt, wherein the ammonium is selected from the group consisting of quaternary ammonium, the conjugate acid of a basic amino acid, acylic or cyclic aliphatic ammonium, heterocyclic ammonium, aromatic ammonium, substituted and unsubstituted pyridinium, guanidinium, and amidinium.

In some embodiments, the IPM analog used herein has a formula of IIa (Scheme II). In some embodiments, the IPM analog salt used herein has a formula of II (Scheme II), wherein A⁺ is defined herein.

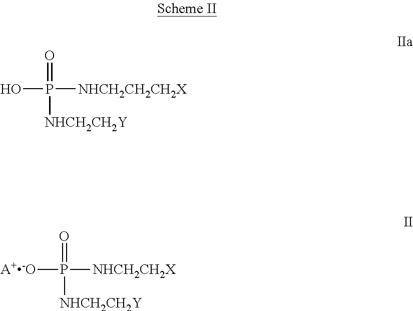

In some embodiments, ranges are expressed herein as from “about” one particular value, and/or to “about” another particular value. When such a range is expressed, another embodiment includes from the one particular value and/or to the other particular value. Similarly, when values are expressed as approximations, such as by use of the antecedent “about,” it is understood that the particular value forms another embodiment. It may be further understood that the endpoints of each of the ranges are significant both in relation to the other endpoint, and independently of the other endpoint.

In this specification and in the claims which follow, reference will be made to a number of terms which shall be understood to have the following meanings:

“Optional” or “optionally” means that the subsequently described event or circumstance can but need not occur, and that the description includes instances where said event or circumstance occurs and instances where it does not.

The term “amino acid” refers to both natural and unnatural amino acids, including without limitation α-amino acids, in their D and L stereoisomers for chiral amino acids. Examples of basic amino acid residues include those having a basic side chain, such as an amino or guanidino group. Basic amino acid residues include, without limitation, arginine, histidine, homoarginine, lysine, homolysine and ornithine.

The term “antibody” means an immunoglobulin, whether natural or wholly or partially synthetically produced. All derivatives thereof which maintain specific binding ability are also included in the term. The term also covers any protein having a binding domain which may be homologous or largely homologous to an immunoglobulin binding domain. These proteins may be derived from natural sources, or partly or wholly synthetically produced. Antibodies used herein may be monoclonal or polyclonal.

As used herein, “aliphatic amine” refers to a compound of the formula NR¹R²R³, wherein at least one of R¹⁻³ is an aliphatic group. The term “acyclic aliphatic amine” refers to an aliphatic amine as above, wherein the aliphatic groups are acyclic. The term “heterocyclic amine” refers to a compound of the formula NR¹R²R³, wherein at least one of R¹⁻³ is a heterocyclic group or R¹, R² and/or R³ taken together with their common nitrogen atom form a ring.

As used herein, “subject” refers to a human or an animal.

In one aspect, a method for treating a subject having a hyperproliferative disorder is described, including administering to the subject a composition including:
IPM, an IPM analog, or a pharmaceutically acceptable salt thereof in the dosage of from about 70 mg/m²/day to about 160 mg/m²/day;
etoposide in the dosage from about 50 mg/m²/day to about 130 mg/m²/day; and
one or more DNA cross-linking agent(s) selected from the group consisting of carboplatin, cisplatin, oxaliplatin, and a combination thereof in the dosage of from about AUC 2 mg/mL/min to about AUC 7 mg/mL/min;
   where the treatment does not result in a dose limiting toxicity.

In any one of the preceding embodiments, the dosage of IPM, the IPM analog, or the pharmaceutically acceptable salt thereof is from about 80 mg/m²/day to about 130 mg/m²/day.

In any one of the preceding embodiments, the etoposide dosage is from 90 mg/m²/day to about 100 mg/m²/day.

In any one of the preceding embodiments, the one or more DNA cross-linking agent(s) is carboplatin.

In any one of the preceding embodiments, carboplatin is administered in the dosage of about AUC 4 mg/mL/min.

In any one of the preceding embodiments, the IPM salt is an ammonium salt, where the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

In any one of the preceding embodiments, the IPM salt is IPM tris(hydroxymethyl)methylammonium salt.

In any one of the preceding embodiments, the hyperproliferative disorder is lung cancer.

In any one of the preceding embodiments, the hyperproliferative disorder is one or more diseases selected from the group consisting of small cell lung cancer, non-small cell lung cancer, ovarian cancer, primary mediastinal nonseminomatous germ cell tumor, and a combination thereof.

In any one of the preceding embodiments, the subject has a partial response as defined by RECIST 1.1 in the range of about 30% to about 60%.

In any one of the preceding embodiments, the dose limiting toxicity is a dose limiting neurotoxicity, nephrotoxicity, or hemotoxicity.

In any one of the preceding embodiments, the subject has an Eastern Cooperative Oncology Group (ECOG) performance status of 0, 1, or 2.

In any one of the preceding embodiments, the subject has an Eastern Cooperative Oncology Group (ECOG) performance status of 2.

In any one of the preceding embodiments, the one or more DNA cross-linking agent(s) is carboplatin administered in the dosage of about AUC 4 mg/mL/min.

In another aspect, a method for treating a subject having a hyperproliferative disorder with an Eastern Cooperative Oncology Group (ECOG) performance status of 2 or better at the start of treatment is described, including administering to the subject a composition including:
IPM, an IPM analog, or a pharmaceutically acceptable salt thereof;
etoposide; and
one or more DNA cross-linking agent(s) selected from the group consisting of carboplatin, cisplatin, oxaliplatin, and a combination thereof;
   wherein the treatment does not result in dose limiting toxicity in the subject.

In any one of the preceding embodiments, the dosage of IPM, the IPM analog, or the pharmaceutically acceptable salt thereof is from about 70 mg/m²/day to about 160 mg/m²/day;
the dosage of etoposide is from about 50 mg/m²/day to about 130 mg/m²/day; and
the dosage of the one or more DNA cross-linking agent(s) is from about AUC 2 mg/mL/min to about AUC 7 mg/mL/min.

In any one of the preceding embodiments, the dosage of the IPM salt is from about 80 mg/m²/day to about 130 mg/m²/day.

In any one of the preceding embodiments, the dosage of the etoposide is from about 90 mg/m²/day to about 130 mg/m²/day.

In any one of the preceding embodiments, the dosage of the one or more DNA cross-linking agent(s) is from about AUC 2 mg/mL/min to about AUC 4 mg/mL/min.

In any one of the preceding embodiments, the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

In any one of the preceding embodiments, the IPM salt is IPM tris(hydroxymethyl)methylammonium salt.

In any one of the preceding embodiments, the hyperproliferative disorder is lung cancer.

In any one of the preceding embodiments, the hyperproliferative disorder is one or more diseases selected from the group consisting of small cell lung cancer, non-small cell lung cancer, ovarian cancer, primary mediastinal nonseminomatous germ cell tumor, and a combination thereof.

In any one of the preceding embodiments, the subject has a partial response as defined by RECIST 1.1 in the range of about 30% to about 60%.

In yet another aspect, a method for treating a subject having a hyperproliferative disorder is described, including administering to the subject in a dosing cycle:
IPM, an IPM analog, or a pharmaceutically acceptable salt thereof on days 1, 2, and 3 of the dosing cycle;
etoposide on days 1, 2, and 3 of the dosing cycle; and
one or more DNA cross-linking agent(s) selected from the group consisting of carboplatin, cisplatin, oxaliplatin, and a combination thereof on day 1 of the dosing cycle.

In any one of the preceding embodiments, the treatment does not result in dose limiting toxicity in the subject.

In any one of the preceding embodiments, the dosage of the IPM salt is from about 70 mg/m²/day to about 160 mg/m²/day;
the dosage of etoposide is from about 50 mg/m²/day to about 130 mg/m²/day; and
the dosage of the one or more DNA cross-linking agent(s) is from about AUC 2 mg/mL/min to about AUC 7 mg/mL/min.

In any one of the preceding embodiments, the dosage of the IPM salt is from about 80 mg/m²/day to about 130 mg/m²/day.

In any one of the preceding embodiments, the dosage of etoposide is from about 90 mg/m²/day to about 130 mg/m²/day.

In any one of the preceding embodiments, the dosage of the one or more DNA cross-linking agent(s) is from about AUC 2 mg/mL/min to about AUC 4 mg/mL/min.

In any one of the preceding embodiments, the one or more DNA cross-linking agent(s) is carboplatin administered in the dosage of about AUC 4 mg/mL/min.

In any one of the preceding embodiments, the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

In any one of the preceding embodiments, the IPM salt is IPM tris(hydroxymethyl)methylammonium salt.

In any one of the preceding embodiments, the hyperproliferative disorder is lung cancer.

In any one of the preceding embodiments, the hyperproliferative disorder is one or more diseases selected from the group consisting of small cell lung cancer, non-small cell lung cancer, ovarian cancer, primary mediastinal nonseminomatous germ cell tumor, and a combination thereof.

In any one of the preceding embodiments, the subject has a partial response as defined by RECIST 1.1 in the range of about 30% to about 60%.

In any one of the preceding embodiments, the dosing cycle has a length of 10, 20, 21, 25, or 30 days.

In any one of the preceding embodiments, the subject has an Eastern Cooperative Oncology Group (ECOG) performance status of 0, 1, or 2.

In any one of the preceding embodiments, the subject has an Eastern Cooperative Oncology Group (ECOG) performance status of 2.

In any one of the preceding embodiments, the one or more DNA cross-linking agent(s) is carboplatin administered in the dosage of about AUC 4 mg/mL/min.

In yet another aspect, a method for treating a subject having a hyperproliferative disorder is described, including:
determining an AUC dosage of one or more DNA cross-linking agent(s) to be administered using Calvert formula or Chatelut formula; and
administering to the subject:
   IPM, an IPM analog, or a pharmaceutically acceptable salt thereof;
   etoposide; and
   the one or more DNA cross-linking agent(s) in the determined dosage;
where the one or more DNA cross-linking agent(s) is selected from the group consisting of carboplatin, cisplatin, oxaliplatin, and a combination thereof.

In any one of the preceding embodiments, the determined dosage for the one or more DNA cross-linking agent(s) is from about AUC 2 mg/mL/min to about AUC 7 mg/mL/min.

In any one of the preceding embodiments, the determined dosage for the one or more DNA cross-linking agent(s) is about AUC 4 mg/mL/min.

In any one of the preceding embodiments, the one or more DNA cross-linking agent(s) is carboplatin.

In any one of the preceding embodiments, the treatment does not result in dose limiting toxicity in the subject.

In any one of the preceding embodiments, the dosage of the IPM salt is from about 70 mg/m²/day to about 160 mg/m²/day; and the dosage of etoposide is up to about 100 mg/m²/day.

In any one of the preceding embodiments, the dosage of the IPM salt is from about 80 mg/m²/day to about 130 mg/m2/day.

In any one of the preceding embodiments, the dosage of etoposide is from about 50 mg/m²/day to about 130 mg/m²/day.

In any one of the preceding embodiments, the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

In any one of the preceding embodiments, the IPM salt is IPM tris(hydroxymethyl)methylammonium salt.

In any one of the preceding embodiments, the hyperproliferative disorder is lung cancer.

In any one of the preceding embodiments, the hyperproliferative disorder is one or more diseases selected from the group consisting of small cell lung cancer, non-small cell lung cancer, ovarian cancer, primary mediastinal nonseminomatous germ cell tumor, and a combination thereof.

In any one of the preceding embodiments, the subject has a partial response as defined by RECIST 1.1 in the range of about 30% to about 60%.

In any one of the preceding embodiments, the subject has an Eastern Cooperative Oncology Group (ECOG) performance status of 0, 1, or 2.

In any one of the preceding embodiments, the subject has an Eastern Cooperative Oncology Group (ECOG) performance status of 2.

In any one of the preceding embodiments, the one or more DNA cross-linking agent(s) is carboplatin administered in the dosage of about AUC 4 mg/mL/min.

In any one of the preceding embodiments, the IPM salt is IPM tris(hydroxymethyl)methylammonium salt administered in the dosage of about 130 mg/m²/day, the dosage of carboplatin is about AUC 4 mg/mL/min, and the dosage of etoposide is about 100 mg/m²/day.

In yet another aspect, a method for treating a subject having a hyperproliferative disorder using a 21-day dosing cycle is described, including administering to the subject a composition including:
IPM, an IPM analog, or a pharmaceutically acceptable salt thereof in the dosage of about 130 mg/m²/day on days 1, 2, and 3 of the 21-day dosing cycle;
etoposide in the dosage of about 100 mg/m²/day on days 1, 2, and 3 of the 21-day dosing cycle; and
carboplatin in the dosage of about AUC 4 mg/mL/min on day 1 of the 21-day dosing cycle.

In any one of the preceding embodiments, the subject has an Eastern Cooperative Oncology Group (ECOG) performance status of 0, 1, or 2.

In any one of the preceding embodiments, the subject has an Eastern Cooperative Oncology Group (ECOG) performance status of 2.

In any one of the preceding embodiments, the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

In any one of the preceding embodiments, the IPM salt is IPM tris(hydroxymethyl)methylammonium salt.

In any one of the preceding embodiments, the method further comprises delaying the administration of IPM, the IPM analog, or the pharmaceutically acceptable salt thereof upon the occurrence of one or more adverse events.

In any one of the preceding embodiments, the administration of IPM, the IPM analog, or the pharmaceutically acceptable salt thereof is delayed for 1, 2, 3, 4, 5, 6, 7, 8, 9, 10, 11, 12, 13, 14, 15, 16, 17, 18, 19, 20, or 21 day(s).

In any one of the preceding embodiments, the method further comprises reducing the dosage of IPM, the IPM analog, or the pharmaceutically acceptable salt thereof upon the occurrence of one or more adverse events.

In any one of the preceding embodiments, the dosage of IPM, the IPM analog, or the pharmaceutically acceptable salt thereof is reduced by about 10%, 15%, 20%, 25%, 30%, 35%, 40%, 45%, or 50%.

In any one of the preceding embodiments, the dosage of IPM, the IPM analog, or the pharmaceutically acceptable salt thereof is reduced by about 20%-25%, 25%-30%, 30%-35%, 35%-40%, 40%-45%, or 45%-50%.

In any one of the preceding embodiments, the adverse event is a Grade 4, a Grade 3, or a Grade 2 adverse event.

In any one of the preceding embodiments, the adverse event is one or more events selected from the group consisting of a hematologic adverse event, Glomerular Filtration Rate (eGFR) reduction, and a non-hematologic adverse event.

In yet another aspect, use of a composition is described, including:
IPM, an IPM analog, or a pharmaceutically acceptable salt thereof in the dosage of from about 70 mg/m²/day to about 160 mg/m²/day;
etoposide in the dosage from about 50 mg/m²/day to about 130 mg/m²/day; and
one or more of carboplatin, cisplatin, oxaliplatin, or a combination thereof in the dosage of from about AUC 2 mg/mL/min to about AUC 7 mg/mL/min;
in the manufacture of a medicament to treat a subject having a hyperproliferative disorder.

In any one of the preceding embodiments, the subject has an Eastern Cooperative Oncology Group (ECOG) performance status of 0, 1, or 2.

In any one of the preceding embodiments, the subject has an Eastern Cooperative Oncology Group (ECOG) performance status of 2.

In any one of the preceding embodiments, the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

In any one of the preceding embodiments, the IPM salt is IPM tris(hydroxymethyl)methylammonium salt.

### BRIEF DESCRIPTION OF THE DRAWINGS

A more complete appreciation of the present invention and many of its advantages will be understood by reference to the following detailed description when considered in connection with the following drawing, which is presented for the purpose of illustration only and is not intended to be limiting, and in which:

FIG. 1 illustrates the response of a subject with primary mediastinal nonseminomatous germ cell tumor after treatment with the combination therapy according to one or more embodiments disclosed herein.

### DETAILED DESCRIPTION

The following explanations of terms and examples are provided to better describe the present compounds, compositions and methods, and to guide those of ordinary skill in the art in the practice of the present disclosure. It is also understood that the terminology used in the disclosure is for the purpose of describing particular embodiments and examples only and is not intended to be limiting.

In one aspect, a method of treating a subject with a hyperproliferative disorder is described, including administering to the subject a composition including IPM, an analog thereof, or a pharmaceutically acceptable salt thereof; etoposide and/or teniposide; and one or more DNA cross-linking agent(s) selected from the group consisting of one or more of carboplatin, cisplatin, oxaliplatin, picoplatin, and a combination thereof.

In some embodiments, the hyperproliferative disorders treated according to the disclosed method include those characterized by abnormal cell growth and/or differentiation, such as cancers and other neoplastic conditions. Typical examples of hyperproliferative disorders that can be treated using the disclosed methods, compounds and compositions are listed below. In some specific embodiments, Teniposide is used in the composition described herein for the treatment of childhood leukemia.

Examples of hematological tumors that can be treated using the methods, compounds and compositions disclosed herein include leukemias including acute leukemia (such as acute lymphocytic leukemia, childhood leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Additional examples of conditions that can be treated using the disclosed compounds and compositions include solid tumors, such as sarcomas and carcinomas, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

In some specific embodiments, the hyperproliferative disorder is lung cancer. In some specific embodiments, the hyperproliferative disorder is germ cell tumor. Non-limiting examples of germ cell tumors include testicular pure seminomas, non-seminomatous germ cell tumors, and mixed seminoma tumors. In some specific embodiments, the hyperproliferative disorder is one or more diseases selected from the group consisting of small cell lung cancer, non-small cell lung cancer, ovarian cancer, primary mediastinal nonseminomatous germ cell tumor, and a combination thereof. In some embodiments, the hyperproliferative disorder is an extensive disease lung cancer, e.g., extensive disease non-small cell lung cancer. In some embodiments, the hyperproliferative disorder is chemotherapy naïve. In some embodiments, the hyperproliferative disorder is treatment naïve, extensive disease lung cancer, e.g., treatment naïve extensive disease non-small cell lung cancer.

In some embodiments, the IPM analog used herein has a formula of IIa (Scheme II). In some embodiments, the IPM analog salt used herein has a formula of II (Scheme II).

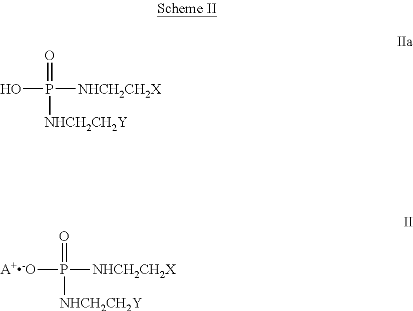

The IPM analogs and salts thereof as described herein include compounds disclosed in international application number PCT/US2005/03 8523, the content of which is incorporated by reference. The IPM analogs and salts thereof as described herein also include compounds disclosed in international application number PCT/US2008/004449, the content of which is incorporated by reference. The IPM analogs and salts thereof as described herein also include compounds and compositions disclosed in international application number PCT/US2009/052295, the content of which is incorporated by reference.

In Scheme II, X and Y represent leaving groups known in the art. Without limitation to theory, it may be believed that the two leaving groups are displaced in vivo by biomolecular nucleophiles, such as nucleic acids and proteins, thereby cross-linking the biomolecules. The term “leaving group” refers to a group that can be displaced by a nucleophile or a molecular fragment that departs with a pair of electrons in heterolytic bond cleavage. Leaving groups can be anions or neutral molecules. With reference to the presently disclosed compounds, leaving group refers to a group that can be displaced to form an aziridinium intermediate, or can be directly displaced by a biomolecular nucleophile, such as a nucleic acid nucleophile, to form, for example, an alkylated guanidinium species. Examples of suitable leaving groups include the halides such as Cl⁻, Br⁻, and I⁻, and sulfonate esters, such as para-toluenesulfonate (“tosylate”, TsO⁻). In one embodiment of the disclosed IPM analog salts, the compound may be a “mixed” leaving group compound, including two different types of leaving groups, for example a halogen and a sulfonate or two different halogens, such as a bromide and a chloride. U.S. Pat. No. 6,197,760 to Struck teaches methods for making such mixed leaving group compounds, which is incorporated by reference.

In Scheme II, A⁺ represents an ammonium salt, wherein the ammonium may be selected from the group consisting of quaternary ammonium, the conjugate acid of a basic amino acid, acylic or cyclic aliphatic ammonium, heterocyclic ammonium, aromatic ammonium, substituted and unsubstituted pyridinium, guanidinium, and amidinium. In some embodiments, the IPM analogs and salts are crystalline as described in international application number PCT/US2008/004449.

In some specific embodiments, IPM (Formula Ia) or a salt thereof (Formula I) may be used in the composition for treatment of the subject with a hyperproliferative disorder, wherein A⁺ is defined herein. In some specific embodiments, the IPM salt used is IPM tris(hydroxymethyl)methylammonium salt.

In some specific embodiments, the subject may be administered IPM, an IPM analog, or a pharmaceutically acceptable salt thereof in the dosage from about 70 mg/m²/day to about 160 mg/m²/day, from about 70 mg/m²/day to about 150 mg/m²/day, from about 70 mg/m²/day to about 140 mg/m²/day, from about 70 mg/m²/day to about 130 mg/m²/day, from about 70 mg/m²/day to about 120 mg/m²/day, from about 70 mg/m²/day to about 110 mg/m²/day, from about 70 mg/m²/day to about 100 mg/m²/day, from about 70 mg/m²/day to about 90 mg/m²/day, or from about 70 mg/m²/day to about 80 mg/m²/day.

In some specific embodiments, the subject may be administered IPM, an IPM analog, or a pharmaceutically acceptable salt thereof in the dosage from about 80 mg/m²/day to about 160 mg/m²/day, from about 80 mg/m²/day to about 150 mg/m²/day, from about 80 mg/m²/day to about 140 mg/m²/day, from about 80 mg/m²/day to about 130 mg/m²/day, from about 80 mg/m²/day to about 120 mg/m²/day, from about 80 mg/m²/day to about 110 mg/m²/day, from about 80 mg/m²/day to about 100 mg/m²/day, or from about 80 mg/m²/day to about 90 mg/m²/day.

In some specific embodiments, the subject may be administered IPM, an IPM analog, or a pharmaceutically acceptable salt thereof in the dosage from about 90 mg/m²/day to about 160 mg/m²/day, from about 90 mg/m²/day to about 150 mg/m²/day, from about 90 mg/m²/day to about 140 mg/m²/day, from about 90 mg/m²/day to about 130 mg/m²/day, from about 90 mg/m²/day to about 120 mg/m²/day, from about 90 mg/m²/day to about 110 mg/m²/day, or from about 90 mg/m²/day to about 100 mg/m²/day.

In some specific embodiments, the subject may be administered IPM, an IPM analog, or a pharmaceutically acceptable salt thereof in the dosage from about 100 mg/m²/day to about 160 mg/m²/day, from about 100 mg/m²/day to about 150 mg/m²/day, from about 100 mg/m²/day to about 140 mg/m²/day, from about 100 mg/m²/day to about 130 mg/m²/day, from about 100 mg/m²/day to about 120 mg/m²/day, or from about 100 mg/m²/day to about 110 mg/m²/day.

In some specific embodiments, the subject may be administered IPM, an IPM analog, or a pharmaceutically acceptable salt thereof in the dosage from about 110 mg/m²/day to about 160 mg/m²/day, from about 110 mg/m²/day to about 150 mg/m²/day, from about 110 mg/m²/day to about 140 mg/m²/day, from about 110 mg/m²/day to about 130 mg/m²/day, or from about 110 mg/m²/day to about 120 mg/m²/day.

In some specific embodiments, the subject may be administered IPM, an IPM analog, or a pharmaceutically acceptable salt thereof in the dosage from about 120 mg/m²/day to about 160 mg/m²/day, from about 120 mg/m²/day to about 150 mg/m²/day, from about 120 mg/m²/day to about 140 mg/m²/day, or from about 120 mg/m²/day to about 130 mg/m²/day.

In some specific embodiments, the subject may be administered IPM, an IPM analog, or a pharmaceutically acceptable salt thereof in the dosage from about 130 mg/m²/day to about 160 mg/m²/day, from about 130 mg/m²/day to about 150 mg/m²/day, or from about 130 mg/m²/day to about 140 mg/m²/day.

In some specific embodiments, the subject may be administered IPM, an IPM analog, or a pharmaceutically acceptable salt thereof in the dosage from about 140 mg/m²/day to about 160 mg/m²/day, or from about 140 mg/m²/day to about 150 mg/m²/day.

In some specific embodiments, the subject may be administered IPM, an IPM analog, or a pharmaceutically acceptable salt thereof in the dosage from about 125 mg/m²/day to about 130 mg/m²/day, from about 125 mg/m²/day to about 135 mg/m²/day, or from about 130 mg/m²/day to about 145 mg/m²/day. In some embodiments, dosages may be less than about 70 mg/m²/day or more than about 160 mg/m²/day as not all embodiments of this disclosure are intended to be limited in this respect.

In some specific embodiments, the subject may be administered IPM, an IPM analog, or a pharmaceutically acceptable salt thereof in the dosage of about 70, 75, 80, 85, 90, 95, 100, 105, 110, 115, 120, 125, 130, 135, 140, 145, 150, 155, or 160 mg/m²/day. In a specific embodiment, IPM tris(hydroxymethyl)methylammonium salt with a dosage of 130 mg/m²/day may be used in the combination therapy.

Applicants have surprisingly found that up to about 130 mg/m²/day of IPM, an IPM analog, or a pharmaceutically acceptable salt thereof can be administered, in combination with etoposide and/or teniposide and one or more of carboplatin, cisplatin, and oxaliplatin, to a subject having a hyperproliferative disorder, without resulting in a dose limiting toxicity in the subject. Generally, a dose limiting toxicity (DLT) is where the appearance of side effects during treatment is severe enough to prevent further increase in dosage or strength of treatment agent, or to prevent continuation of treatment at any dosage level. For example, in some embodiments, the DLT is where the appearance of side effects during treatment requires a patient being treated to discontinue treatment prior to a scheduled discontinuation or break in treatment. As used herein, the DLT is defined in the Experimental section and is related to Grade 3 or higher adverse effect as defined by the National Cancer Institute (NCI) Common Terminology Criteria for Adverse Events (CTCAE) v4.03. See, http://evs.nci.nih.gov/ftp1/CTCAE/About.html and the definitions of Grades of adverse effect herein. Severe Adverse Effect (SAE) includes neurotoxicity, nephrotoxicity, or hemotoxicity. In some embodiments, the DLT refers to adverse effect (AE) occurred in the 1^{st} dosing cycle only.

As used herein, the severity of each AE will be determined by using the NCI's Common Terminology Criteria for Adverse Events (NCI-CTCAE), version 4.03 (available at: http://evs.nci.nih.gov/ftp1/CTCAE/About.html) as a guideline whenever possible. Grade refers to the severity of the adverse event. In those cases where the NCI-CTCAE does not apply, intensity should be defined according to the following criteria:
1. Grade 1: Mild; asymptomatic or mild symptoms; clinical or diagnostic observations only; intervention not indicated.
2. Grade 2: Moderate; minimal, local or noninvasive intervention indicated; limiting age appropriate instrumental Activities of Daily Living (ADL).
3. Grade 3: Severe or medically significant but not immediately life threatening; hospitalization or prolongation of hospitalization indicated; disabling; limiting self-care ADL.
4. Grade 4: Life-threatening consequences; urgent intervention indicated.
5. Grade 5: Death related to AE.

In some embodiments, the subject suitable for treatment with the methods and compositions described herein has an Eastern Cooperative Oncology Group (ECOG) performance status of 0, 1, 2, or more. Because of the toxic effects of carboplatin and etoposide (CE) combination therapy, the combination regimen including carboplatin and etoposide (CE) is typically not recommended for a subject with a Eastern Cooperative Oncology Group (ECOG) performance status of at least 2 at the start of treatment. In contrast, the combination therapy disclosed herein has a reduced and improved toxicity profile and can be used to treat such subjects with an ECOG performance status of 2 or better. In some embodiments, a subject with an ECOG performance status of at least 2 can be treated with compositions described herein including carboplatin in the dosage of about AUC 4 mg/mL/min. Subjects with an ECOG performance status of 0 or 1 can also be treated with the combination therapy disclosed herein.

The IPM, analog, or salt combination therapy with etoposide and/or teniposide and platinum agents described herein results in surprisingly good safety profile with marked efficacy. In some embodiments, the subject after treatment has a partial response as defined by RECIST 1.1 in the range of about 30% to about 60%. See, Eisenhauer et al., “New response evaluation criteria in solid tumors: revised RECIST guideline (version 1.1),” Eur. J. Cancer 2009, 45, 228-247. In some specific embodiments, the partial response may be more than about 30%, about 40%, about 50%, about 60%.

Etoposide and/or teniposide may be used in the combination therapy disclosed herein. The etoposide or teniposide dosage may be from about 50 to about 130 mg/m²/day. In some embodiments, the etoposide or teniposide dosage may be up to or about 100 mg/m²/day. In some embodiments, the etoposide or teniposide dosage may be up to or about 100 mg/m²/day. In some embodiments, the etoposide or teniposide dosage may be about 50, 60, 70, 80, 85, 90, 95, 100, 105, 110, 120, or 130 mg/m²/day. In some embodiments, the etoposide or teniposide dosage may be in the range of about 70-80 mg/m²/day, 70-85 mg/m²/day, 70-90 mg/m²/day, 70-95 mg/m²/day, 70-100 mg/m²/day, 70-105 mg/m²/day, 70-110 mg/m²/day, 70-115 mg/m²/day, 70-120 mg/m²/day, 80-85 mg/m²/day, 80-90 mg/m²/day, 80-95 mg/m²/day, 80-100 mg/m²/day, 80-105 mg/m²/day, 80-110 mg/m²/day, 80-115 mg/m²/day, 80-120 mg/m²/day, 85-90 mg/m²/day, 85-95 mg/m²/day, 85-100 mg/m²/day, 85-105 mg/m²/day, 85-110 mg/m²/day, 85-115 mg/m²/day, 85-120 mg/m²/day, 90-95 mg/m²/day, 90-100 mg/m²/day, 90-105 mg/m²/day, 90-110 mg/m²/day, 90-115 mg/m²/day, 90-120 mg/m²/day, 95-100 mg/m²/day, 95-105 mg/m²/day, 95-110 mg/m²/day, 95-115 mg/m²/day, 95-120 mg/m²/day, 100-105 mg/m²/day, 100-110 mg/m²/day, 100-115 mg/m²/day, 100-120 mg/m²/day, 105-110 mg/m²/day, 105-115 mg/m²/day, 105-120 mg/m²/day, 110-115 mg/m²/day, or 115-120 mg/m²/day.

Additionally, one or more DNA cross-linking agent(s) selected from the group consisting of one or more of carboplatin, cisplatin, and oxaliplatin may be used in the combination therapy disclosed herein. In some embodiments, carboplatin may be used in the combination therapy disclosed herein. In other embodiments, cisplatin may be used in the combination therapy disclosed herein. The dosage of the platinum agent, e.g., cisplatin, carboplatin, oxaliplatin, or a combination thereof, may be from about AUC 2 mg/mL/min to about AUC 7 mg/mL/min, from about AUC 3 mg/mL/min to about AUC 7 mg/mL/min, from about AUC 4 mg/mL/min to about AUC 7 mg/mL/min, from about AUC 5 mg/mL/min to about AUC 7 mg/mL/min, from about AUC 6 mg/mL/min to about AUC 7 mg/mL/min, from about AUC 2 mg/mL/min to about AUC 6 mg/mL/min, from about AUC 3 mg/mL/min to about AUC 6 mg/mL/min, from about AUC 4 mg/mL/min to about AUC 6 mg/mL/min, from about AUC 5 mg/mL/min to about AUC 6 mg/mL/min, from about AUC 2 mg/mL/min to about AUC 5 mg/mL/min, from about AUC 3 mg/mL/min to about AUC 5 mg/mL/min, from about AUC 4 mg/mL/min to about AUC 5 mg/mL/min, from about AUC 2 mg/mL/min to about AUC 4 mg/mL/min, from about AUC 3 mg/mL/min to about AUC 4 mg/mL/min, or from about AUC 2 mg/mL/min to about AUC 3 mg/mL/min,. In some specific embodiments, the dosage of the platinum agent may be about AUC 2 mg/mL/min, about AUC 2.5 mg/mL/min, about AUC 3 mg/mL/min, about AUC 3.5 mg/mL/min, about AUC 4 mg/mL/min, about AUC 4.5 mg/mL/min, about AUC 5 mg/mL/min, about AUC 5.5 mg/mL/min, about AUC 6 mg/mL/min, or about AUC 7 mg/mL/min. In some specific embodiments, carboplatin may be administered in the dosage of about less than about AUC 5.5, about 5, about 4.5, or about 4 mg/mL/min. In some specific embodiments, carboplatin may be administered in the dosage of about AUC 4 mg/mL/min.

Due to the nephrotoxicity associated with cisplatin, AUC-based dosing with carboplatin may be often favored for elderly patients and those with cardiac or renal comorbidity. AUC stands for the area under the curve and is the level of drug exposure in a subject in concentration as a function of time (i.e., mg/mL/min). As used herein, the AUC dosage refers to the dosage of a drug which results in certain value of area under the plasma concentration vs. time curve. For instance, as used herein, a AUC 4 or AUC 4 mg/mL/min dosage refers to the dosage of a drug administered to a subject which results in 4 mg/mL/min area under the curve concentration of the drug. In some embodiments, a AUC dosage of 2-7 is used for carboplatin, cisplatin, oxaliplatin, or a combination thereof in the method of treatment. In certain embodiments, the carboplatin dose in milligrams may be calculated using the Calvert formula: Total carboplatin dose (mg)=Target AUC×(estimated creatinine clearance+25). Creatinine clearance may be estimated using the Cockcroft-Gault equation below for purposes of dosing carboplatin.

GFR=(140-age)*(Wt in kg)*(0.85 if female)/(72*Cr).

In other embodiments, the AUC dosage of carboplatin may be calculated using the Chatelut formula: Total dose (mg)=target AUC(mg/ml/min)×estimated creatinine clearance (ml/min) The Calvert formula may be used with all non-Chatelut formulas for creatinine clearance while the Chatelut formula may be used only with Chatelut formula for carboplatin clearance. The U.S. FDA is recommending a cap of 125 ml/min for the creatinine clearance (regardless of whether it's calculated or measured) in carboplatin dosing.

In some embodiments, the combination therapy used herein includes (i) IPM salt in the dosage from about 80 mg/m²/day to about 130 mg/m²/day; (ii) etoposide in the dosage up to about 100 mg/m²/day; and (iii) carboplatin or cisplatin in the dosage of from AUC 2 mg/mL/min to AUC 7 mg/mL/min. In some specific embodiments, the combination therapy used herein includes (i) IPM tris(hydroxymethyl)methylammonium salt in the dosage of about 130 mg/m²/day; (ii) etoposide in the dosage of about 100 mg/m²/day; and (iii) carboplatin in the dosage of about AUC 4 mg/mL/min. In other specific embodiments, the combination therapy used herein includes (i) IPM tris(hydroxymethyl)methylammonium salt in the dosage of about 130 mg/m²/day; (ii) etoposide in the dosage of about 100 mg/m²/day; and (iii) cisplatin in the dosage of about AUC 4 mg/mL/min.

Suitable dosing cycles known in the art are contemplated. In some embodiments, 1, 2, 3, 4, 5, 6, 7, 8, 9, 10, 11 or more dosing cycles are used. Each dosing cycle may be about 10 days, 20 days, 21 days, 25 days, or 30 or more days. In some embodiments, the dosing cycle may be about 21 days. In each dosing cycle, the combination therapy may be administered to the subject in any of the dosages described herein for a number of days, e.g., 1, 2, 3, 4 or more days, followed by days without any combination therapy administering. In some embodiments, IPM, an IPM analog, or a pharmaceutically acceptable salt thereof may be dosed on day 1 of the dosing cycle. In some embodiments, IPM, an IPM analog, or a pharmaceutically acceptable salt thereof may be dosed only on days 1, 2, and 3 of the dosing cycle. In some embodiments, etoposide and/or teniposide may be dosed on days 1, 2, and 3 of the dosing cycle. In some embodiments, the one or more DNA cross-linking agents selected from the group consisting of carboplatin, cisplatin, oxaliplatin, picoplatin, and a combination thereof may be dosed on day 1 of the dosing cycle. Thus, in some embodiments, an IPM salt may be dosed on days 1, 2, and 3 of a dosing cycle; carboplatin is dosed on day 1 of the dosing cycle; and etoposide may be then dosed on days 1, 2, and 3 of the dosing cycle. In some embodiments, an IPM salt may be dosed on days 1, 2, and 3 of a 21-day dosing cycle; carboplatin may be dosed on day 1 of the dosing cycle; and etoposide may be then dosed on days 1, 2, and 3 of the 21-day dosing cycle. Any suitable dosages as described herein can be used. In some embodiments, the IPM salt may be administered in the dosage up to about 130 mg/m²/day; etoposide may be administered in the dosage up to about 100 mg/m²/day; and the one or more DNA cross-linking agent(s) is administered in the dosage of from about AUC 2 mg/mL/min to about AUC 7 mg/mL/min.

In certain embodiments, the method described herein also includes determining an AUC dosage of one or more DNA cross-linking agent(s) to be administered using Calvert formula described above. The determined AUC dosage of the one or more DNA cross-linking agent(s) may be then used in the method for treating the subject. Thus, the method includes: determining an AUC dosage of the one or more DNA cross-linking agent(s) to be administered using Calvert formula; and administering to the subject: (i) IPM, an IPM analog, or a pharmaceutically acceptable salt thereof; (ii) etoposide and/or teniposide; and (iii) the one or more DNA cross-linking agent(s) in the determined dosage; wherein the one or more DNA cross-linking agent(s) is selected from the group consisting of carboplatin, cisplatin, oxaliplatin, picoplatin, and a combination thereof. In some embodiments, the determined dosage for the one or more DNA cross-linking agent(s) is from about AUC 2 mg/mL/min to about AUC 7 mg/mL/min. In some embodiments, the determined dosage for the one or more DNA cross-linking agent(s) is about AUC 4 mg/mL/min. In some embodiments, the determined dosage for the one or more DNA cross-linking agent(s) is up to about AUC 4 mg/mL/min. In some embodiments, the one or more DNA cross-linking agent(s) may be carboplatin. In some embodiments, the method includes dosing IPM tris(hydroxymethyl)methylammonium salt in the dosage of about 130 mg/m²/day, carboplatin in the dosage of from about AUC 4 mg/mL/min, and etoposide in the dosage of about 100 mg/m²/day.

In yet another aspect, use of a composition including (i) IPM or a pharmaceutically acceptable salt thereof in the dosage of from about 70 mg/m²/day to about 160 mg/m²/day; (ii) etoposide in the dosage from about 50 mg/m²/day to about 130 mg/m²/day; and (iii) one or more of carboplatin, cisplatin, oxaliplatin, or a combination thereof in the dosage of from about AUC 2 mg/mL/min to about AUC 7 mg/mL/min in the manufacture of a medicament to treat a subject having a hyperproliferative disorder is described. In some embodiments, the composition is used for the manufacture of a medicament to treat subject having an Eastern Cooperative Oncology Group (ECOG) performance status of 0, 1, or 2. In some specific embodiments, the composition is used for the manufacture of a medicament to treat subject having an Eastern Cooperative Oncology Group (ECOG) performance status of 2. In some specific embodiments, the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium. In some specific embodiments, the IPM salt is IPM tris(hydroxymethyl)methylammonium salt.

In some embodiments, the dosing of IPM, the IPM analog, or the pharmaceutically acceptable salt thereof may be delayed upon the occurrence of adverse events. In some embodiments, the dosages of IPM, the IPM analog, or the pharmaceutically acceptable salt thereof may be reduced upon the occurrence of adverse events. In some embodiments, the adverse event is a Grade 4 or higher adverse event. In some embodiments, the adverse event is a Grade 3 or higher adverse event. In some embodiments, the adverse event is a Grade 2 or higher adverse event. Non-limiting examples of the adverse events include hematologic adverse events, Glomerular Filtration Rate (eGFR) reduction, and other non-hematologic adverse events. Non-limiting examples of non-hematologic adverse events include cardiac disorders, lymphatic disorders, congenital, familial and genetic disorders, ear and labyrinth disorders, endocrine disorders, eye disorders, gastrointestinal disorders, general disorders and administering site conditions, hepatobiliary disorders, immune system disorders, infections and infestations, injury, poisoning and procedural complications, metabolism and nutrition disorders, musculoskeletal and connective tissue disorders, benign, malignant and unspecified (including cysts and polyps) neoplasms, nervous system disorders, psychiatric disorders, renal and urinary disorders, reproductive system and breast disorders, respiratory, thoracic and mediastinal disorders, skin and subcutaneous tissue disorders, and vascular disorders. Other examples of the adverse events can be found online at http://evs.nci.nih.gov/ftp1/CTCAE/About.html.

In some embodiments, the hematologic adverse events include a reduction of platelet count (e.g., to <100×10⁹/L), a reduction of absolute neutrophil count (e.g., to <1,500/mm³ or <1,000/mm³), neutropenic fever, increased serum amylase (e.g., >5.0× Upper Limit of Normal), hemolysis, and leukocytosis.

In some embodiments, the dosing of IPM, the IPM analog, or the pharmaceutically acceptable salt thereof is delayed for more than 1 day. In some embodiments, the dosing of IPM, the IPM analog, or the pharmaceutically acceptable salt thereof is delayed for more than 2, 3, 4, 5, 6, 7, 8, 9, 10, 11, 12, 13, 14, 15, 16, 17, 18, 19, 20, or 21 days. In some embodiments, the dosing of IPM, the IPM analog, or the pharmaceutically acceptable salt thereof is delayed for 1 day or for 2, 3, 4, 5, 6, 7, 8, 9, 10, 11, 12, 13, 14, 15, 16, 17, 18, 19, 20, or 21 days. In some embodiments, the delay in dosing results in the amelioration of the adverse events. For instance, a Grade 4 adverse event may become a Grade 3, 2, or 1 adverse event after the delay. After the delay, IPM, the IPM analog, or the pharmaceutically acceptable salt thereof may be dosed in regular dosages or reduced dosages.

In some embodiments, upon the occurrence of the adverse events, the dosage of IPM, the IPM analog, or the pharmaceutically acceptable salt thereof is reduced by more than about 20%, 30%, 40%, or 50%. In some embodiments, the reduction in dosage is about 10%, 15%, 20%, 25%, 30%, 35%, 40%, 45%, or 50%. In some embodiments, the reduction in dosage is about 10%-15%, 15%-20%, 20%-25%, 25%-30%, 30%-35%, 35%-40%, 40%-45%, or 45%-50%. In some embodiments, the reduction of dosage of IPM, the IPM analog, or the pharmaceutically acceptable salt thereof is used without the dosing delays of IPM, the IPM analog, or the pharmaceutically acceptable salt thereof to ameliorate the adverse effect. In some embodiments, the reduction of dosage of IPM, the IPM analog, or the pharmaceutically acceptable salt thereof is used in combination with the dosing delays of IPM, the IPM analog, or the pharmaceutically acceptable salt thereof to ameliorate the adverse effect.

In some embodiments, the dosing of etoposide and/or teniposide may be delayed upon the occurrence of the adverse events. In some embodiments, the dosing of the one or more DNA cross-linking agent(s) selected from the group consisting of carboplatin, cisplatin, oxaliplatin, picoplatin, and a combination thereof may be delayed upon the occurrence of the adverse events. In some embodiments, the dosing of etoposide, teniposide and/or the one or more DNA cross-linking agent(s) is delayed for more than 2, 3, 4, 5, 6, 7, 8, 9, 10, 11, 12, 13, 14, 15, 16, 17, 18, 19, 20, or 21 days. In some embodiments, the dosing is delayed for 1 day or for 2, 3, 4, 5, 6, 7, 8, 9, 10, 11, 12, 13, 14, 15, 16, 17, 18, 19, 20, or 21 days. In some embodiments, the delay in dosing results in the amelioration of the adverse events. In some embodiments, the dosages of etoposide, teniposide and/or the one or more DNA cross-linking agent(s) are reduced upon the occurrence of the adverse events. In some embodiments, the dosing delays or dosage reductions of these drugs are determined by the drugs' monographs available locally.

In some embodiments, the dosage reduction and/or dosing delay of IPM, the IPM analog, or the pharmaceutically acceptable salt thereof are used in combination with the dosage reduction and/or dosing delay of etoposide, teniposide and/or the one or more DNA cross-linking agent(s). In other embodiments, the dosage reduction and/or dosing delay of IPM, the IPM analog, or the pharmaceutically acceptable salt thereof are used without the dosage reduction and/or dosing delay of etoposide, teniposide and/or the one or more DNA cross-linking agent(s).

In some embodiments, IPM, the IPM analog, or the pharmaceutically acceptable salt thereof is reconstituted using sodium chloride solutions for the administration in patients. In some embodiments, the sodium chloride solution has a concentration from about 0.5% to about 15%. In some embodiments, the sodium chloride solution has a concentration from about 0.9% to about 14.6%. In some embodiments, the sodium chloride solution has a concentration of about 0.9%, 1.0%, 2.0%, 2.5%, 3.0%, 3.5%, 4.0%, 4.5%, 5.0%, 5.55, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14%, 14.5%, 14.6%, or 15%. In other embodiments, the sodium chloride solution used is purchased from commercially available sources in the United States or other countries where the patients are treated.

In some embodiments, IPM, the IPM analog, or the pharmaceutically acceptable salt thereof is dosed in patients immediately after being reconstituted in the sodium chloride solution to minimize potential drug decomposition in the solution. In some embodiments, IPM, the IPM analog, or the pharmaceutically acceptable salt thereof is dosed within 3 hr, 2.5 hr, 2 hr, 1.5 hr, 1 hr, 0.5 hr after having been reconstituted in the sodium chloride solution.

### EXAMPLES

### Eastern Cooperative Oncology Group (ECOG) Performance Status.

The ECOG Performance Status is defined as illustrated below.

ECOG PERFORMANCE STATUS*

| Grade | ECOG |
|---|---|
| 0 | Fully active, able to carry on all pre-disease performance |
| | without restriction |
| 1 | Restricted in physically strenuous activity but ambulatory and |
| | able to carry out work of a light or sedentary nature, |
| | e.g., light house work, office work |
| 2 | Ambulatory and capable of all self-care but unable to carry out |
| | any work activities. |
| | Up and about more than 50% of waking hours. |
| 3 | Capable of only limited self-care, confined to bed or chair |
| | >50% of waking hours |
| 4 | Completely disabled. Cannot carry on any self-care. Totally |
| | confined to bed or chair. |
| 5 | Dead |

*Oken et al., “Toxicity and Response Criteria of the Eastern Cooperative Oncology Group,” Am. J. Clin. Oncol. 5, 649-55, 1982.

### Adverse Event (AE) Definition

An adverse event is any untoward medical occurrence associated with the use of a drug in humans. Any worsening of a preexisting condition, which is temporally associated with the use of the study drug, is also an AE. Exemplary adverse events include, without limitation: (i) suspected adverse drug reactions; (ii) reactions from study drug overdose, abuse, withdrawal, sensitivity, or toxicity; (iii) significant changes or abnormalities when compared to baseline, in signs, symptoms, clinical laboratory results, or physiological testing (including any worsening of a preexisting condition temporally associated with the use of study drug); or (iv) other untoward medical events, regardless of their relationship to the study drug, such as injury, events that require surgery, accidents, extensions of symptoms, or apparently unrelated illnesses.

### Example 1

### Multicenter Phase Ib Study of the Safety and Efficacy of IPM Plus Carboplatin/Etoposide (PaCE) in Patients with Small Cell Lung Cancer or Other Selected Cancers

A multicenter phase I, open-label, dose-escalation study assessing the safety and efficacy of IPM-tris in combination with carboplatin (C) and etoposide (E) (PaCE regimen) in SCLC and in other cancers in which C+E is considered appropriate was conducted. Tumor responses were assessed by RECIST 1.1 and relevant tumor markers.

### Inclusion Criteria

To be eligible, each subject met each of the following criteria: (i) age >18 years; (ii) subject with documentation of a malignancy scheduled to receive etoposide and carboplatin therapy (including, but not limited to testicular cancer, thymoma, ovarian cancer, osteosarcoma, non-small cell lung cancer, and small cell lung cancer); (iii) ECOG Performance Status of 0 or 1; (iv) adequate bone marrow, liver, and renal function, as assessed by the following laboratory requirements conducted within 14 days prior to dosing; and (v) subjects for whom there is no curative standard therapy.

### ECOG Performance Status

ECOG performance status was assessed at screening, day 1 of each cycle, and at the Post-treatment safety assessment visit.

### Major Exclusion Criteria:

(i) unstable or clinically significant concurrent medical condition that would, in the opinion of the investigator, jeopardize the safety of a subject and/or their compliance with the protocol; (ii) presence or history of any illness or injury to the urinary tract (renal or post-renal) which may make the subject more susceptible to acute renal insufficiency in the case of potential renal adverse events; (iii) active infection requiring systemic antibacterial/antibiotic, antifungal, or antiviral therapy; (iv) subjects who are currently pregnant or nursing; (v) subjects who have received other investigational drugs within 30 days of enrollment in this study; or (vi) subjects who are within 4 weeks of their last chemotherapy treatment.

### Route of Administration

IPM tris(hydroxymethyl)methylammonium salt (IPM-tris) was given by intravenous (IV) infusion over approximately 30 minutes on days 1, 2, and 3 of each 21 day cycle. Each IPM-tris infusion to a subject was completed within 1 hour (and not more than 90 minutes) of dose preparation in the IV bag. Etoposide was given by IV infusion over approximately 60 minutes on days 1, 2, and 3 of each 21 day cycle. Carboplatin was given by IV infusion over approximately 30 minutes on day 1 of each 21 day cycle.

Each vial of IPM-tris was reconstituted with 14.6% sodium chloride for Injection, to a final concentration of 50.0 mg per mL. If the 14.6% sodium chloride diluent was not available, sites used 5.0%, 5.7%, 8.5%, or 0.9% sodium chloride for injection as the reconstitution diluent according to the same reconstitution instructions. Other standard sodium chloride solutions commercially available in other countries can be used to reconstituted IPM-tris. The IV infusion of IPM-tris was completed within 2.5 hours of reconstitution in the vial.

Radio-labeled IPM-tris may be synthesized as well to monitor and study its metabolism in the patient's body.

In the cases where treatment-related adverse events are observed, the dosing of IPM-Tris (Palifosfamide-tris) can be delayed and/or its dosage can be reduced. Table 1 illustrates potential adverse events and corresponding dosing delays and dosage reductions.

TABLE 1
Dose Delays and Reductions for Palifosfamide-tris-related Toxicity¹

| | |
|---|---|
| Adverse Events² | Action and Palifosfamide-tris Dose Reduction |

Hematologic

| | |
|---|---|
| Platelet count <100 × 10⁹/L | Delay chemotherapy dosing up to 21 days until platelet count ≧100 × |
| | 10⁹/L, then reduce dose of palifosfamide-tris by 20%. |
| Absolute Neutrophil Count | For subjects who are not currently being treated with growth factors, |
| (ANC) <1,500/mm³ or | delay chemotherapy dosing up to 21 days until ANC ≧1,500/mm³, |
| <1,000/mm³ | then maintain palifosfamide-tris dose when restarted and add growth |
| | factors. |
| | For subjects who have previously experienced neutropenia and |
| | therefore are already being treated with growth factors, delay |
| | chemotherapy dosing up to 21 days until ANC ≧1500/mm³, then |
| | reduce dose of palifosfamide-tris by 20%. |
| Neutropenic fever | Delay chemotherapy dosing up to 21 days until resolved, then reduce |
| | dose of palifosfamide-tris by 20%. For subjects who are not currently |
| | being treated with growth factors, add growth factors. |
| Any other Grade 4 hematologic | Delay chemotherapy dosing up to 21 days until Grade ≦1, then |
| toxicity: e.g., serum amylase | reduce dose of palifosfamide-tris by 20%. |
| increased to >5.0 × Upper | |
| Limit of Normal (ULN); | |
| Hemolysis, or Leukocytosis. | |

Estimated Glomerular Filtration Rate (eGFR) (Appendix 3)

| | |
|---|---|
| ≧25% up to <50% decrease | Hold chemotherapy dosing until repeat test is performed. Reduce |
| from baseline³ | dose of palifosfamide-tris by 20% if confirmed on repeat test. |
| ≧50% decrease from baseline³ | Repeat test, and if confirmed, discontinue study treatment. |

Non-hematologic (other than eGFR)

| | |
|---|---|
| Grade 2 or 3 if unresponsive to | Delay chemotherapy dosing up to 21 days until Grade ≦1 or baseline, |
| appropriate therapy (except | then reduce dose of palifosfamide-tris by 20%. |
| alopecia)⁴ | |
| Grade 4 | Delay chemotherapy dosing up to 21 days until Grade ≦1 or baseline, |
| | then reduce dose of palifosfamide-tris by 20%. |

¹The investigator is required to consult the carboplatin and etoposide product monographs available in his/her country to make dose modification or discontinuation decisions for those products.
²Treatment-related adverse event severity assessed on Day 1 of treatment cycle.
³Baseline eGFR determined based on the most recent serum creatinine measurement prior to study drug administration (i.e., Cycle 1 Day 1 or Screening). Generally, the dosage of IPM-tris will be not be reduced if the patient's eGFR decreases by less than 25%. However, if the patient has an eGFR right at the threshold, e.g., a threshold of 60, then if that patient's eGFR decreases by less than 25%, e.g., 20%, a Doctor may reduce the patient's dosing.
⁴IPM-tris or IPM-tris, Carboplatin, and Etoposide combination is not expected to treat alopecia/hair loss.

The dosages of IPM-tris can be reduced by 20%, 20-25%, 30%, 40%, or 50% upon the occurrence of the adverse events. Additionally, the dosing cycle can be adjusted to delay the dosing until the adverse events have been ameliorated. In some embodiments, the dosages of IPM-tris are delayed up to 21 days and the patients are then dosed with IPM-tris in the reduced dosages as described herein. In other embodiments, the IPM-tris dosing cycle time is adjusted to allow for extra time for the patients to recover from adverse events. In still other embodiments, the IPM-tris dosing cycle time is kept the same as the cycle time of carboplatin or etoposide.

In some embodiments, the grading of the adverse events is based on CTCAE as described herein. In those cases where the NCI-CTCAE does not apply, intensity should be defined according to the following criteria: Grade 1: Mild; Grade 2: Moderate; Grade 3: Severe or medically significant but not immediately life threatening; Grade 4: Life-threatening consequences; and Grade 5: Death related to adverse events.

The dosages of Carboplatin and/or Etoposide can be reduced as well by 20%, 20-25%, 30%, 40%, or 50% upon the occurrence of the adverse events. The dosage reductions for carboplatin and/or etoposide may be made alone or in addition to the IPM-tris dosage reduction. In some embodiments, the dosages of Carboplatin and/or Etoposide can be reduced or adjusted according to their labels or the local standard practice in dosing these drugs.

The doses of IPM-tris, etoposide, and carboplatin for each subject were calculated by the pharmacist based on the dose level and the subject's body surface area (BSA) on the first day of each cycle. BSA was calculated using their Institutional guidelines for calculating BSA or, the calculation for BSA by the Mosteller formula was used (note: SQRT=square root):

BSA (m²)=([height (cm)×weight (kg)]/3600)^{1/2}

e.g., BSA=SQRT ([cm*kg]/3600); or in inches and pounds:

BSA (m²)=([height (in)×weight (lbs)]/3131)^{1/2}

The same formula was used throughout the study when calculating a subject's BSA. The dose of carboplatin did not exceed 600 mg per infusion.

Dose Limiting Toxicity (DLT)

A DLT was considered an adverse event if it was
Grade 3 non-bone marrow toxicity not resolving to ≦Grade 2 in ≦3 days; or
Grade 4 non-bone marrow toxicity; or
Grade 4 neutropenia or thrombocytopenia not resolving to ≦Grade 3 in ≦1 week; or
Thrombocytopenia with clinically relevant bleeding, or
Grade 4 neutropenia of any duration with fever ≧38.5° C. or new infection.
   Toxicity grades are defined by CTCAE v. 4.03. Nausea and vomiting were only considered a DLT if refractory to anti-emetics. Diarrhea was considered a DLT only if refractory to anti-diarrheal therapy. Alopecia was not considered a DLT. The Medical Monitor, following review with the PI(s), may also identify a toxicity thought to be related to study drug that, at the discretion of the investigator, is thought to warrant withholding the drug due to a DLT. An IPM-tris dosage of 150 mg/m²/day was found to result in DLT. In some embodiments, only events occurring within the first 21 days (Cycle 1) of treatment were considered DLTs.

The maximum tolerable dose (MTD) of IPM-tris when administered in combination with etoposide and carboplatin at fixed doses (90 mg/m² and AUC 4, respectively) was determined An additional dose cohort was also evaluated using an escalated dose level of etoposide (100 mg/m²) in combination with the IPM-tris MTD and carboplatin (AUC 4). Once the recommended doses were determined, this combination regimen was further studied in subjects with small cell lung cancer. The dose escalation cohort schedule was:

| Cohort | IPM-tris | Etoposide | Carboplatin |
|---|---|---|---|
| −1 | 80 mg/m² | 90 mg/m² | AUC 4* |
| 1 | 100 mg/m² | 90 mg/m² | AUC 4* |
| 2 | 130 mg/m² | 90 mg/m² | AUC 4* |
| 3 | 150 mg/m² | 90 mg/m² | AUC 4* |
| 2A | 130 mg/m²** | 100 mg/m² | AUC 4* |

*Dose not to exceed 600 mg per infusion;
**Based on dose escalation cohorts completed prior to Amendment 6, the maximum tolerated dose of IPM-tris was determined to be 130 mg/m² when administered in combination with etoposide 90 mg/m² and carboplatin AUC 4 (Cohort 2).

Results:

A total of 22 patients (11 females, 11 males) with the following tumor diagnoses were treated: small cell lung cancer (SCLC) (7), non-small cell lung cancer (NSCLC) (3), ovarian (3), sarcomas (3), primary mediastinal nonseminomatous germ cell tumor (PMNSGCT) (1) and other cancers (5). Median number of previous chemotherapies were 3 (Range 0-5). Grade 3 and 4 toxicities were reported by 13 patients, including thrombocytopenia (8), hypophosphatemia (4), anemia (4), neutropenia (3), fatigue (2) and vomiting (1). There were no treatment related deaths. The maximum tolerated dose of IPM-tris in combination with etoposide 100 mg/m² and carboplatin AUC 4 was 130 mg/m². Dose limiting toxicity was seen in one patient with neutropenic fever at 150 mg/m² of IPM-tris. Of the 17 evaluable patients, two partial response (PR) in SCLC, one PR in NSCLC, one PR in ovarian cancer, and one PR (by tumor markers as assessed by RECIST 1.1) in PMNSGCT were noted, all in previously treated patients. Six patients had stable disease (SD). These results show that PaCE is a well-tolerated regimen that has activity in various tumors, including SCLC. Two patients withdrew consent.

| Response Assessments | Number of Patients |
|---|---|
| PR | 5 |
| SD | 6 |
| PD | 5 |
| Patient didn't continue past Cycle 1 | 1 (AE), 1 (DLT at 150 mg/m²/day) |

For one patient with SCLC (extensive disease), a partial response was reported with 34% decrease in tumor burden at end of Cycle 2 and 41% at end of Cycle 4. For another patient with SCLC (extensive disease), a partial response was reported with 43% decrease in tumor burden at end of Cycle 2.

For one patient with NSCLC (extensive disease), a partial response was reported with 44% decrease in tumor burden at end of Cycle 2.

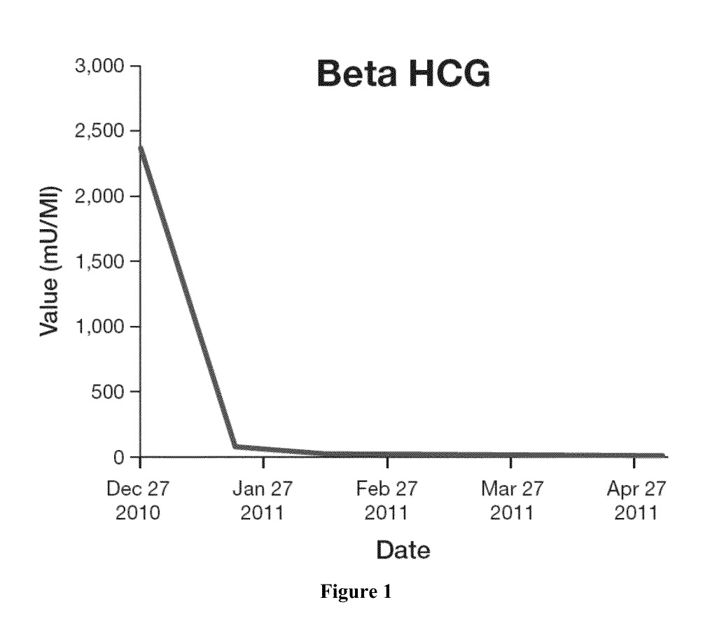

For one patient with Primary Mediastinal Nonseminomatous Germ Cell Tumor, a normalization of tumor markers reported. The level of beta HCG was reduced from 2358 to 12 mU/M1. See, FIG. 1.

For one patient with ovarian cancer, a partial response was reported with a 52% decrease in tumor burden at the end of Cycle 2.

### Example 2

A multi-center, open-label, adaptive, randomized Study of IPM-tris, a novel DNA crosslinker, in combination with carboplatin and etoposide (PaCE) Chemotherapy versus carboplatin and etoposide (CE) alone in chemotherapy naïve patients with extensive-stage Small Cell Lung Cancer is planned. The primary objective of this study is to compare the efficacy of IPM-tris in combination with carboplatin and etoposide (PaCE) chemotherapy to carboplatin and etoposide (CE) alone, as measured by overall survival (OS), in chemotherapy naïve subjects with extensive-stage small cell lung cancer (SCLC). The secondary objectives include: assess secondary efficacy endpoints including time to progression (TTP), objective response rate (ORR), response duration, and effects on quality of life (QOL) and disease-related symptoms; assess potential prognostic factors for OS (i.e., performance status, age, and gender); assess the safety of PaCE chemotherapy in the study population; and collect tumor tissue samples for future analysis of potential biomarkers that may correlate with objective tumor response and/or clinical outcome.

### Study Design

IPM-tris formulation, carboplatin, and etoposide (PaCE) and CE chemotherapy alone will be referred to as “study treatment” throughout this protocol. This study is primarily designed to evaluate the efficacy of PaCE vs. CE chemotherapy as determined by overall survival (OS).

### Inclusion Criteria:

Male or female subjects, age ≧18 years.

Histological or cytological diagnosis of extensive-stage small cell lung cancer.

No prior chemotherapy, adjuvant therapy, or radiotherapy for lung cancer.

Exception: prior radiotherapy for brain metastases is allowed provided that the subject has recovered from any acute treatment related toxicity.

Measurable or non-measurable disease as per RECIST v1.1.

ECOG Performance Status of 0, 1, or 2.

Adequate bone marrow, liver, and renal function, as assessed by the following laboratory requirements:

Male and female subjects of child bearing potential must agree to use a double-barrier method of birth control from the screening visit through 21 days after the last dose of study drug. Specifically, subjects with ECOG Performance Status of 2 are included for this dosing study.

### Dose & Schedule:

IPM-tris (IPM tris(hydroxymethyl)methylammonium salt, 130 mg/m²/day) and etoposide (100 mg/m²/day) will be administered by intravenous (IV) infusion on days 1, 2, and 3 of each 21 day cycle. Carboplatin (target area under the concentration time curve (AUC) 4 mg/mL/min) will be administered by IV infusion on day 1 of each 21 day cycle. The dose of carboplatin does not exceed 600 mg/infusion. A carboplatin dose of AUC 5 was selected for the reference therapy (control) arm within the recommended dose range (AUC 5-6) and considering that the proposed study population will include patients with poor risk factors (e.g., elderly, ECOG performance status 2, or other comorbidity) for whom a higher dose may not be appropriate.

The etoposide dose of 100 mg/m²/day (administered by IV infusion on day 1 of each 21 day cycle) for the CE arm is the same as the recommended dose and matches the PaCE chemotherapy arm, thereby limiting variability and facilitating safety and efficacy comparisons.

The dose (mg) of IPM-tris should be calculated based on the subject's actual body weight as measured on day 1 of each cycle. Body surface area (BSA) may be calculated as per the standard practice at each investigative center, however the same formula should be used throughout the study when calculating a subject's BSA. The IPM-tris dose must be prepared as described in the Pharmacy Manual, including permitted elapsed time following reconstitution until dilution in the IV infusion bag and completion of the IV infusion. Briefly, each vial of IPM-tris will be reconstituted with 5 mL Sodium Chloride (NaCl) for Injection (14.6% NaCl is recommended) and the appropriate dose transferred to a 250 mL 0.9% sodium chloride IV infusion bag. The dose should be administered as an approximately 30 minute IV infusion completed not more than 90 minutes after adding IPM-tris to the infusion bag.

### ECOG Performance Status

The ECOG performance status will be assessed at screening, day 1 of each cycle, and the Post-treatment safety assessment visit.

Those skilled in the art would readily appreciate that all parameters and examples described herein are meant to be exemplary and that actual parameters and examples will depend upon the specific application for which the composition and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. Accordingly, those skilled in the art would recognize that the use of a composition or method in the examples should not be limited as such. The present invention is directed to each individual composition, or method described herein. In addition, any combination of two or more such compositions or methods, if such composition or methods are not mutually inconsistent, is included within the scope of the present invention.

We claim:

1. A method for treating a subject having a hyperproliferative disorder, comprising administering to the subject, in a dosing cycle, a composition comprising:
IPM, an IPM analog, or a pharmaceutically acceptable salt thereof in the dosage of from about 70 mg/m²/day to about 160 mg/m²/day;
etoposide in the dosage from about 50 mg/m²/day to about 130 mg/m²/day; and
one or more DNA cross-linking agents selected from the group consisting of carboplatin, cisplatin, oxaliplatin, and a combination thereof in the dosage of from about AUC 2 mg/mL/min to about AUC 7 mg/mL/min;
wherein the treatment does not result in a dose limiting toxicity.

2. The method of claim 1, wherein dosage of IPM, the IPM analog, or the pharmaceutically acceptable salt thereof is from about 80 mg/m²/day to about 130 mg/m²/day.

3. The method of claim 1, wherein the etoposide dosage is from 90 mg/m²/day to about 100 mg/m²/day.

4. The method of claim 1, wherein the one or more DNA cross-linking agent(s) is carboplatin.

5. The method of claim 4, wherein dosage of carboplatin is about AUC 4 mg/mL/min.

6. The method of claim 1, wherein the IPM salt is an ammonium salt, wherein the ammonium is selected from the group consisting of quaternary ammonium, the conjugate acid of a basic amino acid, acylic aliphatic ammonium, heterocyclic ammonium, aromatic ammonium, substituted and unsubstituted pyridinium, guanidinium, and amidinium.

7. The method of claim 1, wherein the IPM salt is IPM tris(hydroxymethyl)methylammonium salt.

8. The method of claim 1, wherein the hyperproliferative disorder is lung cancer.

9. The method of claim 1, wherein the hyperproliferative disorder is one or more diseases selected from the group consisting of small cell lung cancer, non-small cell lung cancer, ovarian cancer, primary mediastinal nonseminomatous germ cell tumor, and a combination thereof.

10. The method of claim 1, wherein the subject has a partial response as defined by RECIST 1.1 in the range of about 30% to about 60%.

11. The method of claim 1, wherein the dose limiting toxicity is a dose limiting neurotoxicity, nephrotoxicity, or hemotoxicity.

12. The method of claim 1, wherein the subject has an Eastern Cooperative Oncology Group (ECOG) performance status of 0, 1, or 2.

13. The method of claim 1, wherein the subject has an Eastern Cooperative Oncology Group (ECOG) performance status of 2.

14. The method of claim 13, wherein the one or more DNA cross-linking agent(s) is carboplatin administered in the dosage of about AUC 4 mg/mL/min.

15. The method of claim 1, wherein the one or more DNA cross-linking agent(s) is administered in the dosage of from about AUC 2 mg/mL/min to about AUC 4 mg/mL/min.

16. The method of claim 1, wherein
IPM, the IPM analog, or the pharmaceutically acceptable salt thereof is dosed on days 1, 2, and 3 of the dosing cycle;
etoposide is dosed on days 1, 2, and 3 of the dosing cycle; and
the DNA cross-linking agent(s) is dosed on day 1 of the dosing cycle.

17. The method of claim 1, wherein the dosing cycle has a length of 10, 20, 21, 25, or 30 days.

18. The method of claim 1, wherein:
the AUC dosage of the one or more DNA cross-linking agent(s) to be administered is determined by using Calvert formula or Chatelut formula.

19. The method of claim 1, wherein
the dosing cycle is a 21-day dosing cycle,
IPM, the IPM analog, or the pharmaceutically acceptable salt thereof is adminstered on days 1, 2, and 3 of the 21-day dosing cycle;
etoposide is adminstered in the dosage of at about 100 mg/m²/day on days 1, 2, and 3 of the 21-day dosing cycle; and
the one or more DNA cross-linking agent(s) is carboplatin administered in the dosage of about AUC 4 mg/mL/min on day 1 of the 21-day dosing cycle.

20. The method of claim 1, wherein the method further comprises delaying the administration of IPM, the IPM analog, or the pharmaceutically acceptable salt thereof upon the occurrence of one or more adverse events.

21. The method of claim 20, wherein the administration of IPM, the IPM analog, or the pharmaceutically acceptable salt thereof is delayed for 1, 2, 3, 4, 5, 6, 7, 8, 9, 10, 11, 12, 13, 14, 15, 16, 17, 18, 19, 20, or 21 day(s).

22. The method of claim 1, wherein the method further comprises reducing the dosage of IPM, the IPM analog, or the pharmaceutically acceptable salt thereof upon the occurrence of one or more adverse events.

23. The method of claim 20, wherein the method further comprises reducing the dosage of IPM, the IPM analog, or the pharmaceutically acceptable salt thereof upon the occurrence of the one or more adverse events.

24. The method of claim 22, wherein the dosage of IPM, the IPM analog, or the pharmaceutically acceptable salt thereof is reduced by about 10%, 15%, 20%, 25%, 30%, 35%, 40%, 45%, or 50%.

25. The method of claim 22, wherein the dosage of IPM, the IPM analog, or the pharmaceutically acceptable salt thereof is reduced by about 20%-25%, 25%-30%, 30%-35%, 35%-40%, 40%-45%, or 45%-50%.

26. The method of claim 20, wherein the adverse event is a Grade 4, Grade 3, or a Grade 2 adverse event.

27. The method of claim 22, wherein the adverse event is a Grade 4, Grade 3, or a Grade 2 adverse event.

28. The method of claim 20, wherein the adverse event is one or more events selected from the group consisting of a hematologic adverse event, Glomerular Filtration Rate (eGFR) reduction, and a non-hematologic adverse event.

29. The method of claim 22, wherein the adverse event is one or more events selected from the group consisting of a hematologic adverse event, Glomerular Filtration Rate (eGFR) reduction, and a non-hematologic adverse event.
